(19) United States
(12) Patent Application Publication
(10) Pub. No.: US 2011/0306991 A1
(43) Pub. Date: Dec. 15, 2011

(54) ANASTOMOSIS DEVICE AND METHOD OF USING THE SAME

(76) Inventors: Harmanpreet BASSAN, Toronto (CA); Peter KIM, Toronto (CA); Thomas LOOI, Markham (CA)

(21) Appl. No.: 13/157,949

(22) Filed: Jun. 10, 2011

Related U.S. Application Data
(60) Provisional application No. 61/353,993, filed on Jun. 11, 2010.

(51) Int. Cl. A61B 17/04; A61B 17/11

(52) U.S. Cl. 606/144; 606/153

(57) ABSTRACT

The present invention provides an anastomosis device which includes an elongate center pin having proximal and distal ends. The proximal end is configured to be graspable using a grasping tool. A grip mechanism is located at the distal end for crimping a suture. The device includes an outer sleeve having a passageway therethrough to receive therein the elongate center pin. A suture having a suture needle is attached to a first end of the center pin and a second end of the suture is formed into a pre-formed knot mounted on the sleeve. The device includes a suture release mechanism having a passageway extending therethrough to receive the outer sleeve therein. The suture release mechanism slides along the sleeve between a first position in which the suture release mechanism is spaced from the distal end and a second position such that movement of the suture release mechanism from the first position to the second position dislodges the pre-formed knot off the sleeve. A free end of the suture, which was attached to a suture needle but cut lose after the suture has been passed through the tissue being sutured, is crimped by the crimp mechanism. The pre-formed knot is dislodged such that the crimped end of the suture is located within the pre-formed knot and pulling the suture through the knot tightens the pre-formed knot on the suture.

### CROSS REFERENCE TO RELATED U.S PATENT APPLICATION

This patent application relates to U.S. provisional patent application Ser. No. 61/353,993 filed on Jun. 11, 2010, entitled ANASTOMOSIS DEVICE AND METHOD OF USING THE SAME, filed in English, which is incorporated herein in its entirety by reference.

### FIELD OF THE INVENTION

The present invention relates to a device for accomplishing the surgical process of anastomosis i.e. connecting two structures (commonly tubular structures) to restore continuity after resection or to bypass an unresectable disease process. More particularly the present invention relates to an anastomosis device for performing the process of connecting the structures in an end-to-end, side-to-side or an end-to-side fashion.

### BACKGROUND OF THE INVENTION

A common requirement in many surgical procedures is the resection or bypass of a diseased organ. Often the diseased section is a part of a tubular structure i.e. artery, bowel, esophagus and therefore after the resection it is required to reattach the resulting two healthy ends. This procedure is termed as anastomosis and is a fairly easy task to perform in the setting of an open surgery. However, in minimally invasive surgery where the procedure is performed through small incisions in the patient's skin, anastomosis is an extremely difficult skill to learn and execute. Typically, anastomosis time in a MIS procedure range between half an hour up to two hours. Needless to say, the long anastomosis time has a negative impact on the patient due to increased anesthesia requirement. For the surgeon, laparoscopic anastomosis is extremely difficult to learn and perform and is very fatiguing in nature. Increased anastomosis time is also a burden on the healthcare provider as it takes up valuable operating room time and adds to the personnel cost.

U.S. Pat. No. 6,358,258 issued to Arcia et al. discloses an anastomosis device that utilizes multiple flexible needles (designed of Nitinol material) that are deployed through multiple curved guide channels. The design utilizes multiple push rods for actuation and is suitable for end-side type anastomosis.

U.S. Pat. No. 7,029,481 issued to Burdulis et al. discloses an anastomosis device that utilizes multiple needles that are simultaneously is pierced through the tissue using a pneumatic cylinder. The needles latch onto small crimps on the opposite end and pull the sutures through the tissue upon retraction. The other end of the device utilizes multiple flexible needles deployed using curved channels and multiple push rods. The design needs custom needles as the sutures are attached to the distal tip of the needle as opposed to the proximal end found in conventional sutures.

U.S. Patent Application US2008/0275472, to Yossepowitch et al. discloses an anastomosis device that utilizes multiple needle deployment through the use of flexible needle and curved guide channels. The design utilizes multiple push rods and requires custom needles to function. Even though the two ends of the design are attached through a flexible coupler, the design lacks a good suture management scheme and will suffer from suture tangling. Similar to U.S. Pat. No. 7,029,481, the design needs custom needles as the sutures are attached to the distal tip of the needle as opposed to the proximal end found in conventional sutures.

Thus, there is a need for an automated/assisted laparoscopic anastomosis device that can reduce procedure time and operating costs. The device will also be of interest to the surgeons as it would minimize the dependence on a surgeon's dexterity and experience and will reduce the learning curve of this complex task.

### SUMMARY OF THE INVENTION

The present invention provides an anastomosis device, comprising:

a) an elongate center pin having proximal and distal ends, said is proximal end structured to be manipulatable by a manipulation tool, a grip mechanism located at said distal end for gripping a suture;

b) an outer sleeve having a passageway therethrough to receive therein the elongate center pin, said sleeve being mountable by a pre-formed knot formed in a suture; and

c) a suture release mechanism having a passageway extending therethrough to receive the outer sleeve therein, said suture release mechanism being reciprocally translatable on said sleeve between a first position and a second position, and movement of said suture release mechanism from said first position to said second position dislodges said pre-formed knot off said sleeve, and with a free end of said suture being gripped by said grip mechanism, said pre-formed knot is dislodged such that said suture is located within said pre-formed knot and pulling said free end tightens said pre-formed knot on said suture.

In another aspect of the present invention there is provided an anastomosis device, comprising:

a) an elongate center pin having proximal and distal ends, said proximal end structured to be manipulatable by a manipulation tool;

b) an outer sleeve having a passageway therethrough to receive therein the elongate center pin, said sleeve being mountable by a pre-formed knot formed in a suture;

c) a suture release mechanism having a passageway extending therethrough to receive the outer sleeve therein, said suture release mechanism being reciprocally translatable on said sleeve between a first position and a second position, and movement of said suture release mechanism from said first position to said second position dislodges said pre-formed knot off said sleeve, and with a free end of said suture being gripped by said grip mechanism, said pre-formed knot is dislodged such that said suture is located within said pre-formed knot and pulling said free end tightens said pre-formed knot on said suture; and

d) a grip mechanism located at said distal end for gripping a suture, the grip mechanism comprising a head section attached to the distal end of said elongate center pin, and a groove disposed on the head section comprising an elongate channel having a width and length and an opening thereto, wherein the opening has a width less than that of the elongate channel, thereby allowing a suture to be inserted into the channel through the opening and to remain firmly held in place in the channel.

A further understanding of the functional and advantageous aspects of the invention can be realized by reference to the following detailed description and drawings.

### BRIEF DESCRIPTION OF THE DRAWINGS

Embodiments of the invention will now be described, by way of example only, with reference to the drawings, in which:

FIG. 1 shows an exemplary embodiment of two tubular structures after the anastomosis has been performed;

FIG. 2 is a perspective view of an embodiment of an anastomosis device constructed in accordance with the present invention;

FIG. 3 is a side view of the anastomosis device of FIG. 2;

FIG. 4 shows an exploded perspective view of the device of FIG. 2;

FIG. 5 shows a perspective view of a laparoscopic tool that combines grasping and cutting functionality into a single tool used with the anastomosis device;

FIG. 6 shows a close up view of a portion of the grasping and cutting laparoscopic tool of FIG. 5 used with the anastomosis device;

FIG. 7 shows the close up view of a scissor actuation mechanism forming part of the grasping and cutting laparoscopic tool

FIG. 8 shows the preferred embodiment of a laparoscopic tool 200 that combines grasping functionality with a sliding push-rod tool.

FIG. 9 is a close-up view of the distal end of the combined grasper and push-rod tool according to the disclosed invention.

FIG. 10 is a perspective view showing the anastomosis tool with a pre-formed knot;

FIGS. 11 to 14 show various steps carried out by a surgeon using the present anastomosis tool to suturing, in which:

FIG. 11 is a perspective view showing the needle and the suture after they have been manually passed through both sides of the target vessel by the anastomosis device;

FIG. 12 is a perspective view showing the anastomosis device with a combined laparoscopic grasper and push-rod tool, with this configuration the surgeon wraps the trimmed end of the suture around a center pin head forming part of the anastomosis device preferably one or more revolutions;

FIG. 13 is a perspective view showing the pre-formed knot after it has been released from the anastomosis device onto the trimmed end of the suture;

FIG. 14 is a perspective view showing the target vessel after the knot shown released in FIG. 13 has been tightened and trimmed; and

FIG. 15 is a perspective view of a second embodiment in accordance with the present invention.

### DETAILED DESCRIPTION OF THE INVENTION

Without limitation, the majority of the systems described herein are directed to an anastomosis device for performing the process of connecting the structures in an end-to-end, side-to-side or an end-to-side fashion, and method of using the same. As required, embodiments of the present invention are disclosed herein. However, the disclosed embodiments are merely exemplary, and it should be understood that the invention may be embodied in many various and alternative forms.

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to an anastomosis device for performing the process of connecting the structures in an end-to-end, side-to-side or an end-to-side fashion.

As used herein, the term “about”, when used in conjunction with ranges of dimensions, temperatures or other physical properties or characteristics is meant to cover slight variations that may exist in the upper and lower limits of the ranges of dimensions so as to not exclude embodiments where on average most of the dimensions are satisfied but where statistically dimensions may exist outside this region. For example, in embodiments of the present invention dimensions of components of a laparoscopic anastomosis device are given but it will be understood that these are not meant to be limiting.

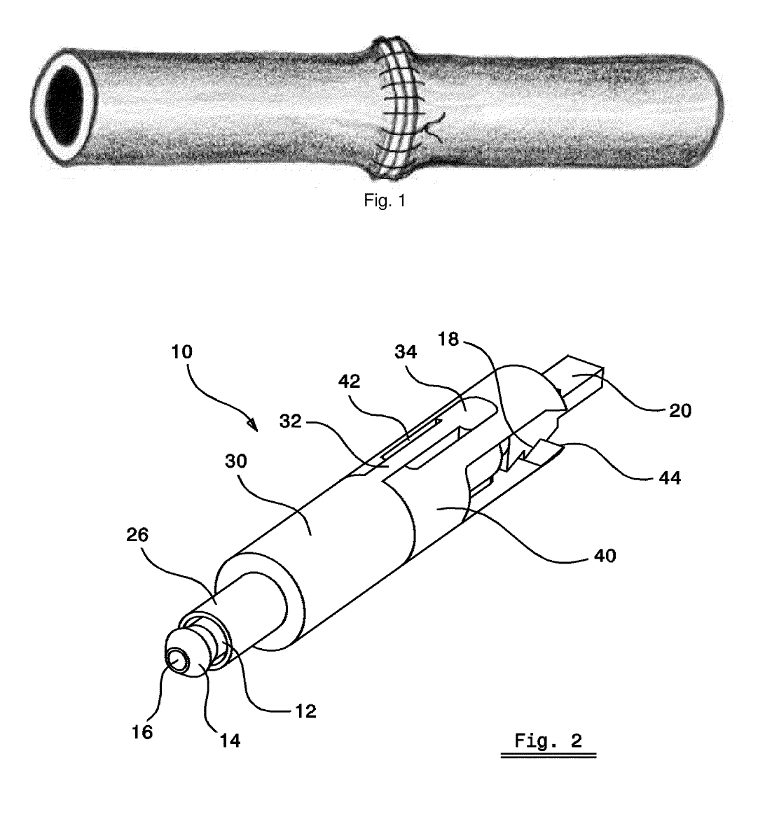

As used herein, the process of “anastomosis” refers to the process of reattaching two healthy ends of an anatomical tubular structure (such as blood veins, arteries, intestines etc.) after a resection has been carried out to remove a diseased or injured section. FIG. 1 shows an exemplary embodiment of two tubular structures after the anastomosis has been performed.

As used herein, the phrase “anastomosis device” refers to a device for performing process of anastomosis which forms the subject matter of the present invention.

The present invention discloses an anastomosis device that utilizes conventional sutures with a mechanism that holds a pre-formed knot to expedite the task of anastomosis. The workflow of the anastomosis device is similar to that of conventional suturing, however it facilitates easy anastomosis that is quick to perform and minimizes the dependence on a surgeon's dexterity.

FIG. 2 shows an embodiment of the anastomosis device 10 comprising four main components: center pin 12, outer sleeve 26, suture release mechanism 30 and locking mechanism 40. The center pin 12 includes a head 14 rigidly attached to it on the distal end through the use of a press fit pin 16. The outer sleeve 26 and locking mechanism 40 are rigidly attached together and therefore act as a single entity in the mechanism. The proximal end of center pin 12 includes saw tooth profile 18 that includes one or more teeth and a proximal end 20 that can be grabbed using a grasper or a similar tool. The center pin 12 can be translated with respect to the outer sleeve 26 through the use of locking mechanism 40. The locking mechanism 40 includes a saw-tooth profile 44 that mates with the saw-tooth profile 18 on the center pin 12. The suture release mechanism 30 can be translated with respect to the locking mechanism 40 and is limited in its motion through the use of built in grooves 42 and a stop 34 in the locking mechanism 40.

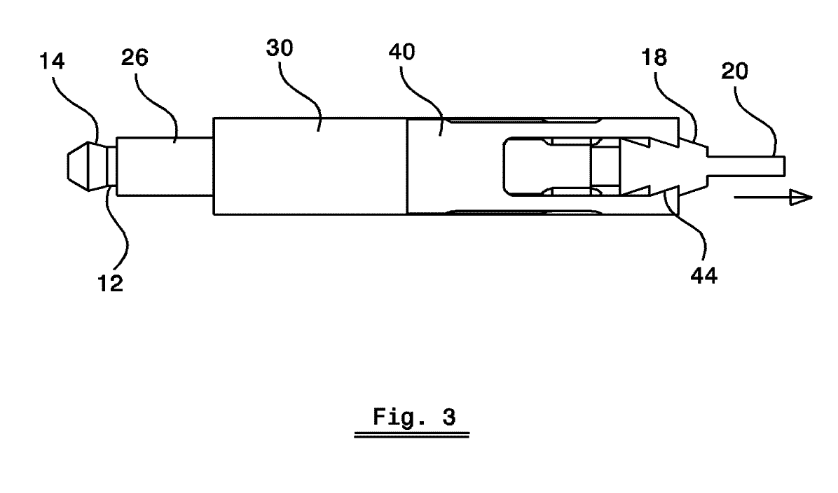

FIG. 3 shows the side view of the embodiment of FIG. 2. Note that the saw-tooth profile 18 on the center pin is designed to mate with the saw-tooth profile 44 on the locking mechanism 40. The locking mechanism 40 permits motion of the center pin 12 in the direction of arrow (as shown) and locks in the opposite direction. Since the device is a one-time use device there is no need to unlock it. At the end of its travel, the center pin 12 is butted against the outer sleeve 26 and is kept in tight contact due to the saw tooth profile 18. In this position, the V-shaped profile on the center pin head 14 is in firm contact with the mating profile on the inside of the outer sleeve 26. The functionality of this profile will be explained in detail later.

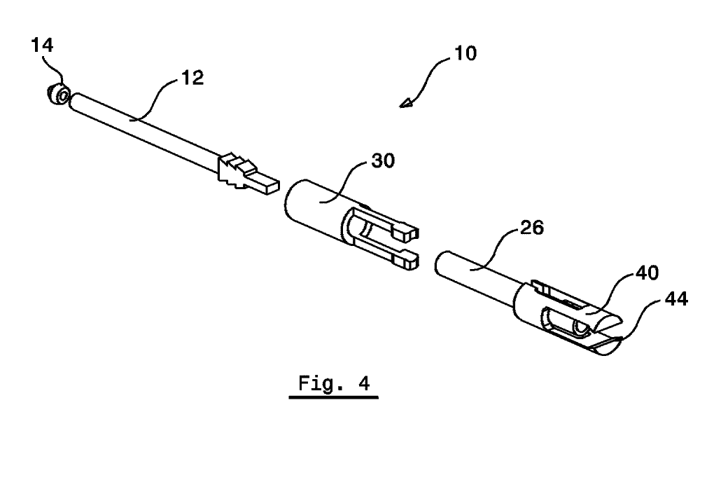

FIG. 4 shows the exploded view of the same embodiment as that shown in FIGS. 2-3. As shown, the center pin includes two parts; center pin body 12 and center pin tip 14 for ease of assembly. The two parts are rigidly attached to each other through the use of a press fit steel pin 16 during assembly.

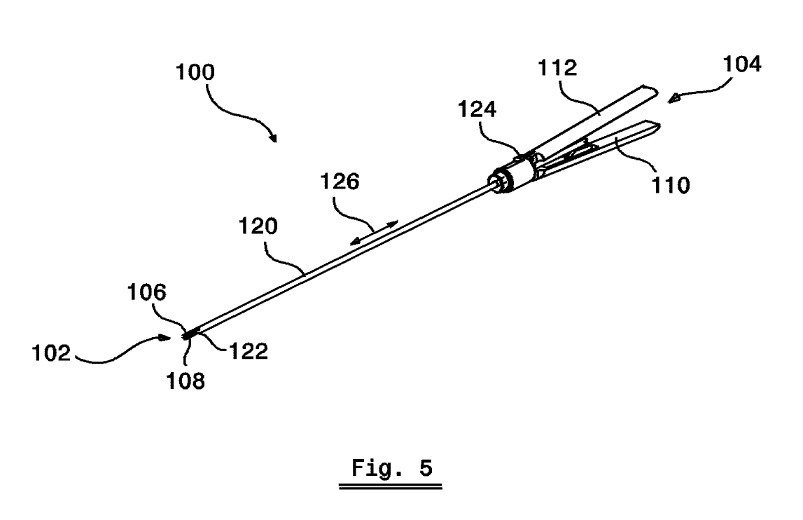

FIG. 5 shows an embodiment of a laparoscopic tool 100 that combines grasping and cutting functionality into a single tool. The tool 100 includes a slender grasping/cutting end 102 that is inserted into the patient's body cavity and a proximal end 104 that includes two levers 110 and 112 for the grasping action and a cutting (scissor) actuation mechanism 124. As with a conventional laparoscopic grasper, the opening and closing of the grasper levers 110 and 112 causes either one or both grasper tips 106 and 108 to open and close, respectively. The scissor actuation mechanism 124 is preferably utilized while the grasper tips 106 and 108 are in its closed position. The forward motion (towards the distal end) of the scissor actuation mechanism 124 causes the outer sleeve 120 of the instrument to move in a forward direction and vice versa.

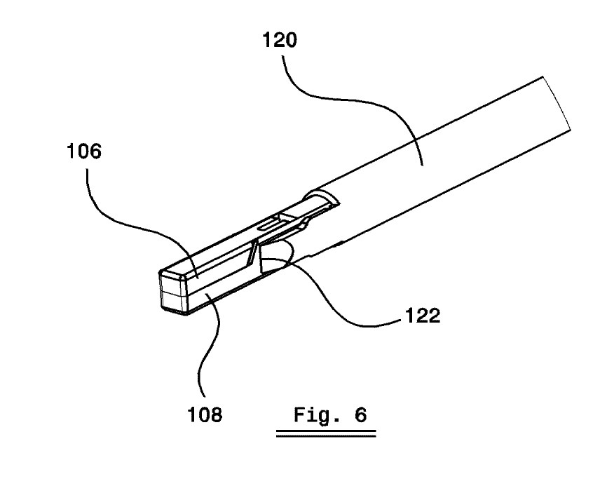

FIG. 6 shows the close up view of the embodiment of a combined grasping and cutting laparoscopic tool 100. Utilizing this tool, the suture can be grasped using the graspers 106 and 108 in a conventional manner. The scissor actuation mechanism 124 can then be translated that will result in the translation of sleeve 120 and therefore scissor edge 122 along the grasper surface, resulting in cutting of the suture.

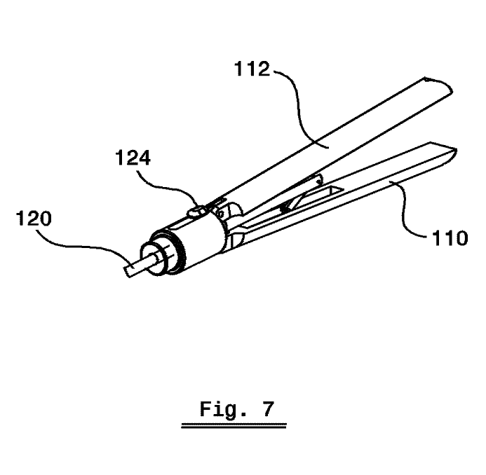

FIG. 7 shows the close up view of the scissor actuation mechanism 124. The forward (towards the distal end of the laparoscopic instrument) motion of the lever 124 causes the scissor tip 122 to translate forward against the grasper surface and results in cutting of the suture.

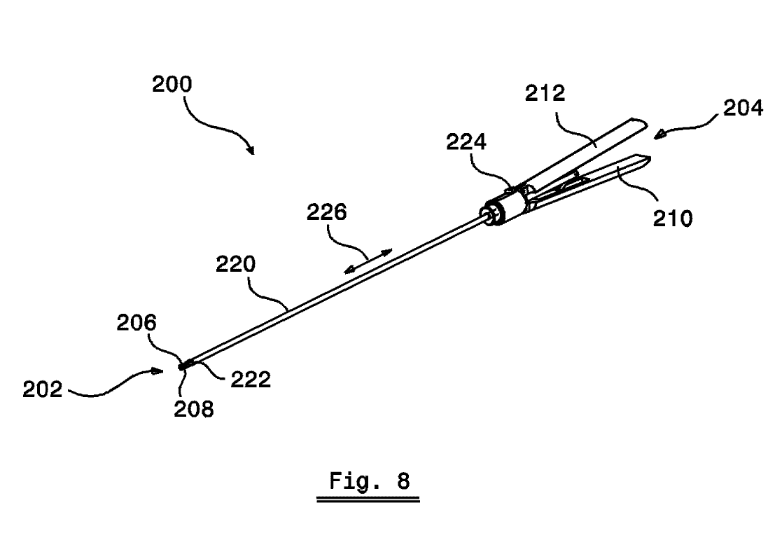

FIG. 8 shows an embodiment of a laparoscopic tool 200 that combines grasping functionality with a sliding push-rod tool. The tool 200 includes a slender end 202 that is inserted into the patient's body cavity and a proximal end 204 that includes two levers 210 and 212 for grasping action and an actuation mechanism 224 for the push-rod. As with a conventional laparoscopic grasper, opening and closing of the grasper levers 210 and 212 causes one or both grasper tips 206 and 208 to open and close, respectively. The push-rod actuation mechanism 224 is utilized while the grasper tips 206 and 208 are in its closed position. The forward motion (towards the distal end) of the push-rod actuation mechanism 224 causes the outer sleeve 220 (and push-rod tip 222) of the instrument to move in a forward direction. The preferred method of use for this tool will be described in details later.

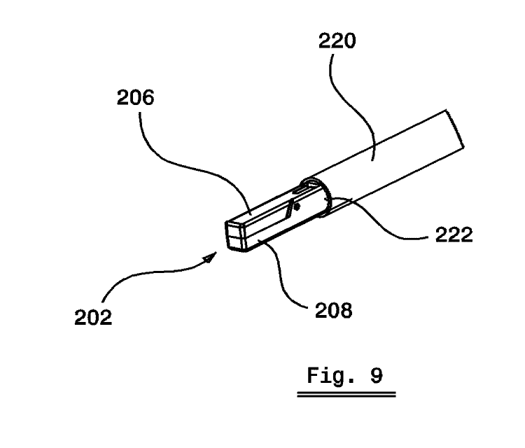

FIG. 9 shows a close-up view of the distal end 202 of the combined grasper and push-rod tool 200.

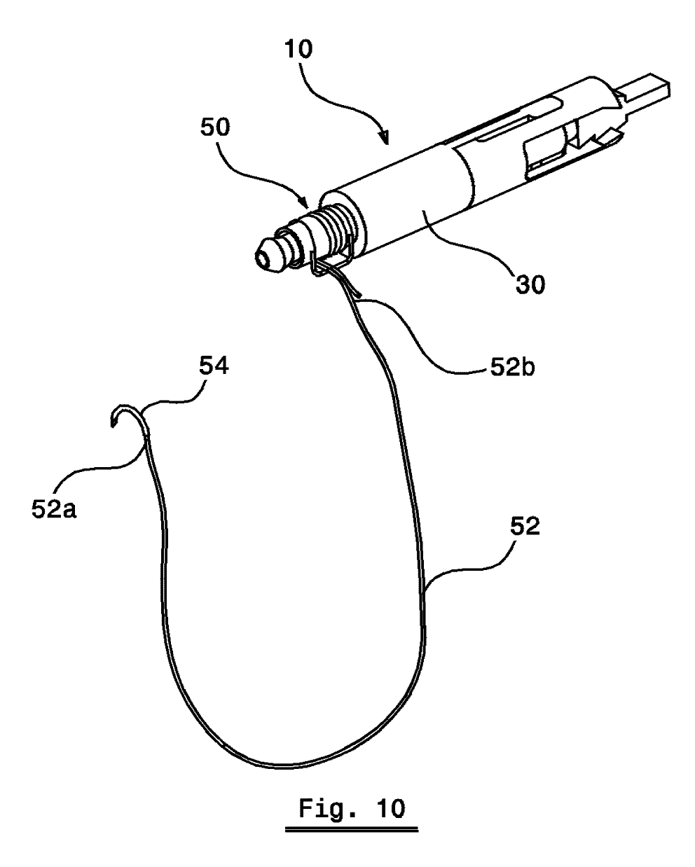

FIG. 10 shows the anastomosis tool 10 with a suture 52 and a pre-formed knot 50 formed on the proximal end 52b of the suture. Knot 50 is preferably a type of slip knot that once slid on suture 52, is prone to unwinding. It can be seen that the anastomosis device 10 is designed as a one-time-use disposable device and is supplied with the needle 54 and suture 52 with a pre-formed knot 50. Various means can be provided on the device to ensure that knot does not accidently slip from the device during handling. One preferred method will be to provide a groove on the suture release mechanism 30 and the proximal end 52b of the suture 52 can be latched in this groove to avoid accidental slippage of the knot. The suture can then be removed from this groove by the surgeon using the laparoscopic grasper after the needle has been passed through both ends of the target anatomy and the anastomosis device is ready to be deployed. Other means of constraining the knot can be utilized without changing the scope of the invention. The preferred method of performing an anastomosis according to disclosed invention will now be described in detail.

One method of performing anastomosis using the disclosed embodiments herein requires the use of two laparoscopic ports on the patient's body. A third laparoscopic port is utilized to obtain images of the target anatomy using an endoscope that is connected to an external monitor for visualization. As a first step, the surgeon introduces the anastomosis device 10 (including the suture 52 and needle 54) into the patient's body cavity through one of the ports. For this task, the surgeon can utilize a laparoscopic tool 100 or 200 to aid in easy insertion. As a next step, the surgeon introduces the combined laparoscopic grasping and cutting tool 100 (FIG. 5) and the combined laparoscopic grasping and push-rod tool 200 (FIG. 8) through the two ports into the patient's body cavity. In a manner similar to conventional anastomosis, the surgeon then utilizes both hands and left and right graspers 100 and 200 to pass the needle through two ends of the organs to be connected.

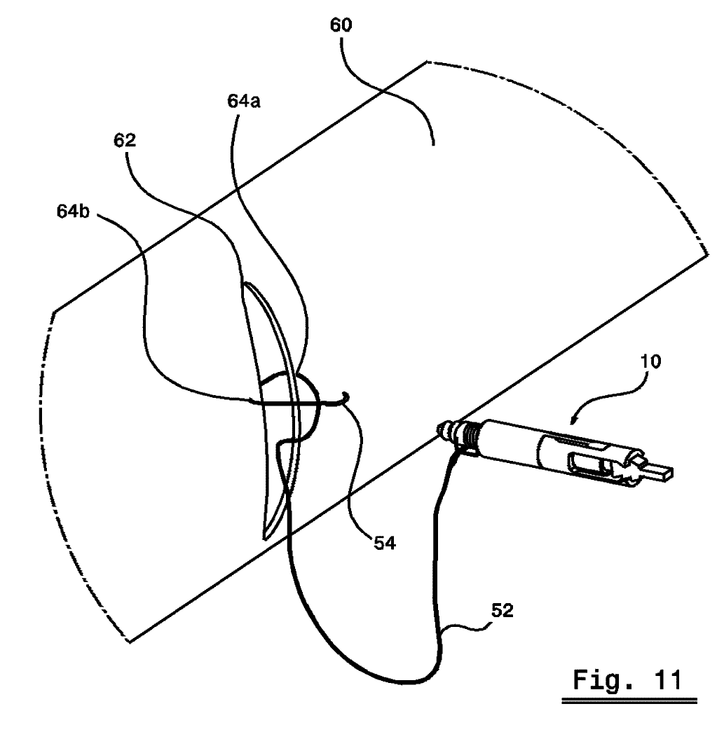

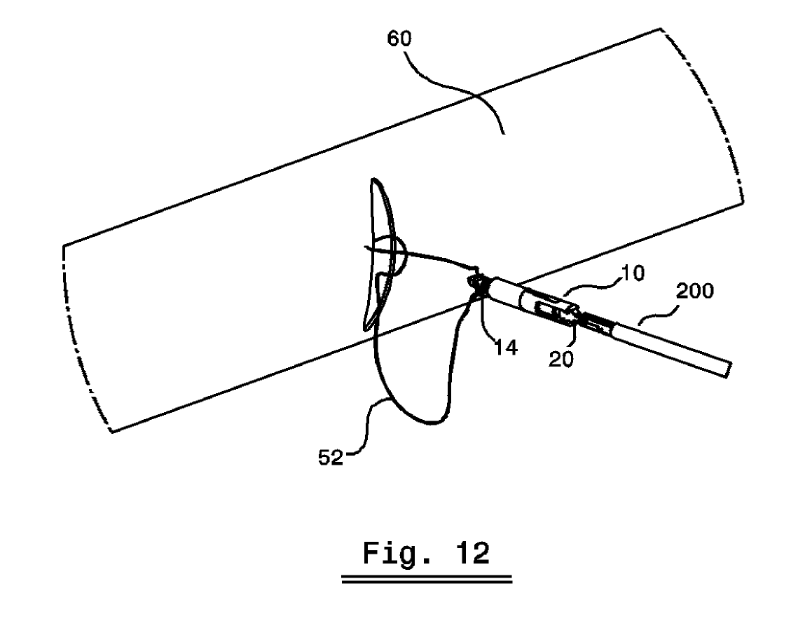

FIG. 11 shows the needle and the suture after they have been manually passed through both sides of the target vessel 60. FIG. 11 also shows an exemplary opening 62 in vessel 60. The objective of anastomosis is to approximate vessel 60 such that the two sides 64a and 64b of the opening 62 are in firm contact with each other and form a leak-proof seal after the anastomosis has been completed. After passing the needle through two sides 64a and 64b, the surgeon utilizes the laparoscopic tools 100 and 200 and the scissor mechanism 122 (on the combined grasper and cutting tool 200) to cut needle 54 from the distal end 52a of suture 52. Optionally, the surgeon can remove needle 54 from the body through one of the laparoscopic ports at this time.

Referring now to FIG. 12, the next step requires the surgeon to grasp the disclosed anastomosis device 10 (from the proximal end 20) using the combined laparoscopic grasper and push-rod tool 200. Using the other hand and the laparoscopic grasper/cutter 100, the surgeon wraps the trimmed end of the suture 52 around the center pin head 14 preferably one or more revolution. With the wrapped suture in its place around the center pin head 14, the surgeon actuates the push-rod mechanism 222 that grips the trimmed end of the suture between the center pin head 14 and the outer sleeve 26. The locking action of the saw-tooth profile 18 and 44 on the center pin 12 and the locking mechanism 40 ensures that the suture 52 stays tightly gripped between the center pin head 14 and the outer sleeve 26. With the center pin 12 firmly held by the laparoscopic grasper and push-rod tool 200, the surgeon utilizes the other laparoscopic tool 100 to actuate the suture release mechanism 30 (by applying a sliding force towards the distal end of the disclosed anastomosis device 10). The suture release mechanism 30 can have notched profile on its surface to aid in application of the proper sliding force. This step essentially slides the pre-formed knot 50 from the outer sleeve 26 on to the trimmed section of the suture 52 (that has already been passed through the target vessel 60).

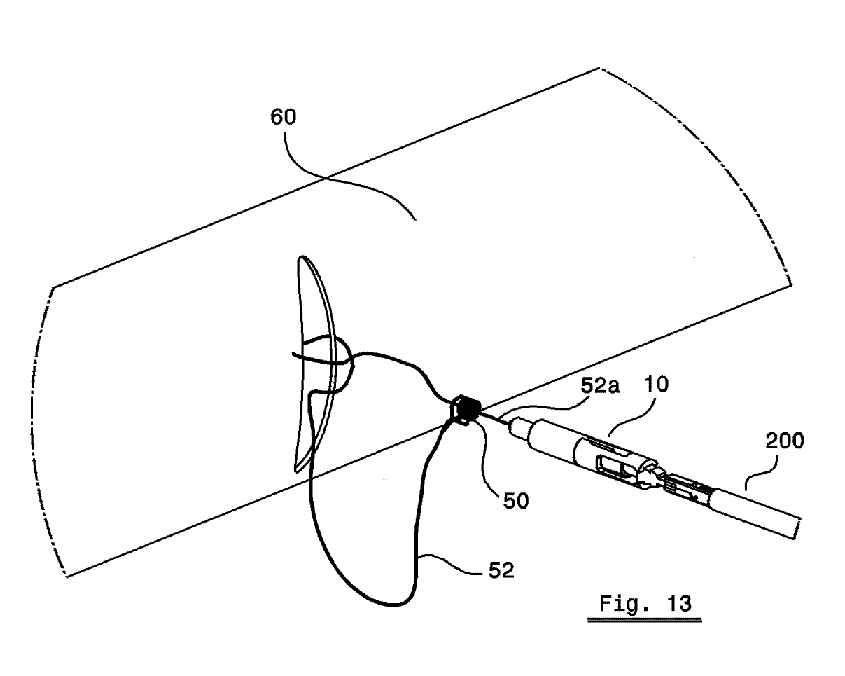

FIG. 13 shows the pre-formed knot 50 after it has been released onto the trimmed end 52a of the suture 52. The surgeon can now release the anastomosis device 10 from the laparoscopic grasper/push-rod tool 200. The surgeon can then utilize both graspers 100 and 200 to tighten the knot 50 on the trimmed section 52a of the suture. The diameter of the outer sleeve 26 is preferably twice the diameter of the suture 52 and therefore knot 50 is fairly well formed as it is released from the outer sleeve 26. From this point onwards, the surgeon can utilize one grasper (for example 200) to hold the trimmed end 52a of the suture 52 and the other grasper 100 to slide the knot 50 towards the target vessel 60 to tighten the knot and complete the anastomosis. Once the wound closure is tight enough (as judged by the surgeon), the surgeon can utilize the cutting tool 122 to trim the suture 52 to the desired length and remove the disclosed anastomosis device 10 (which is also holding the remaining suture 52 gripped on the center pin 12) from the patient's body. Optionally, the surgeon may remove needle 54 from the body through one of the laparoscopic ports at this time.

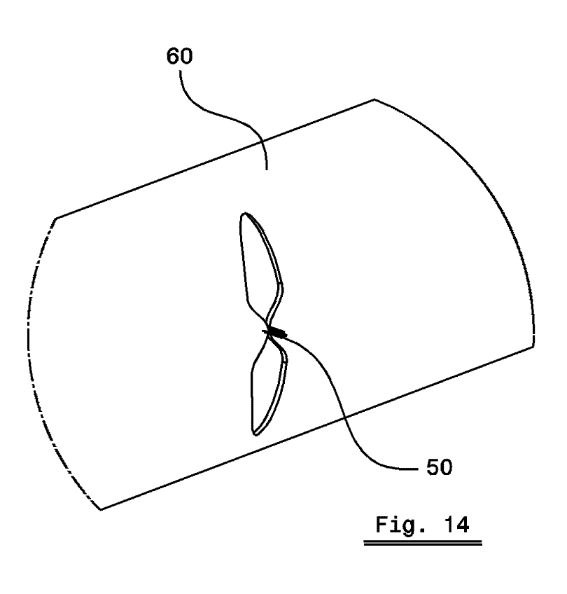

FIG. 14 shows the target vessel 60 after the knot 50 has been tightened and trimmed. These steps are repeated with a new anastomosis device 10 introduced each time and a new knot 50 being applied until a proper anastomosis of the anatomy is achieved. A typical end-to-end anastomosis could require 6 to 8 sutures for proper approximation of the anatomy. In embodiments herein, the task of releasing pre-formed knot from the outer sleeve requires the surgeon to use both hands. However, this task can also be completed with one hand through redesign of the laparoscopic grasper/push-rod 200 through inclusion of another concentric shaft to actuate suture release mechanism 30 without changing the scope of the invention. In addition to the knot 50 shown in the disclosed invention, other forms of knots can be utilized without changing the scope of the invention.

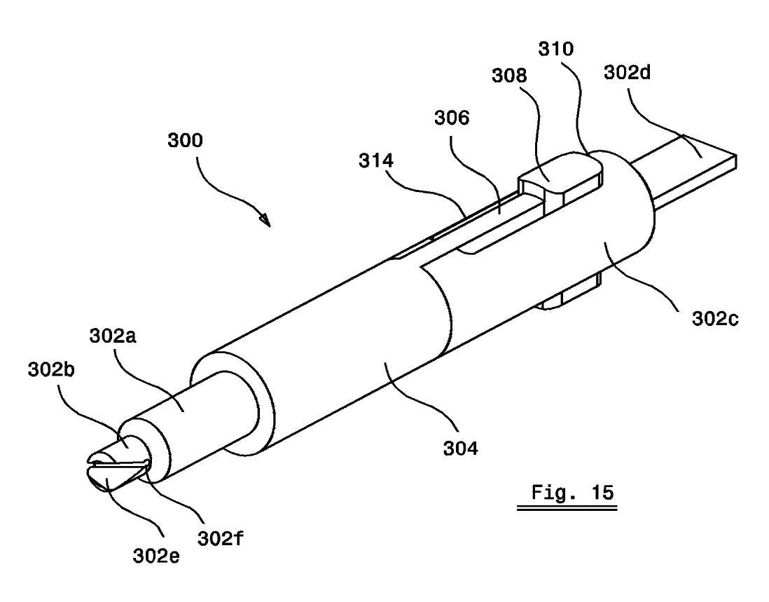

FIG. 15 is a perspective view of a further embodiment of the anastomosis device 300. FIG. 15 shows this embodiment, which includes two components 302 (composed of profiles 302a-302f and 314) and 304. Profile 302a on the distal end is designed to host a pre-formed knot 50 (similar to FIG. 10), profile 302c includes two guiding grooves 314 and profile 302d is the flatted proximal end that can be held utilizing tool 200 (FIG. 8). The distal end also includes a profile 302b (preferably cylindrical) that consists of an opening 302e and a gripping mechanism with a groove 302f. This gripping mechanism is designed to allow a suture to be lockably placed therein. Component 304 includes two substantially narrow profiles 306, and two end-stops 308. Each of the end-stops 308 is designed to slidably fit inside the corresponding guiding groove 314 and can translate linearly with component 304.

It can be seen that device 300 has substantially simplified design as compared to device 10 (FIG. 2) at the same time exhibiting similar functionality. The functioning of device 300 will now be described in detail. Similar to device 10, device 300 is also packaged as one-time use disposable with the suture 52 and is introduced into the patient's body cavity in a similar fashion. Once the distal end 52a of the suture (containing needle 54) has been passed through two ends of the anatomy, the needle 54 is trimmed using the laparoscopic tool 100. The surgeon then utilizes tool 200 and firmly holds device 300 at profile 302d using graspers 206 and 208. Once the device is firmly held, the surgeon utilizes tool 100 to hold free end 52a of suture 52 and inserts it into the gripping mechanism groove 302f. The insertion of suture 52 into groove 302f is assisted by the chamfered profile 302e. The gripping mechanism groove 302f is dimensioned to lockably contain the suture once it is positioned inside the groove. The surgeon then actuates push-rod 220 on tool 200 that causes push-rod tip 222 to linearly translate and come in contact with surface 310 (on end-stop 308). As the push-rod is actuated further, a distal acting force on surface 310 causes component 304 to translated with respect to component 302 and causes preformed knot 50 to slide off profile 302a. The surgeon then releases the grasper from proximal end 302d and utilizes graspers on tools 100 and 200 to tighten the knot. The process is repeated for each anastomosis device 300 until a proper anastomosis is accomplished. It can be noted that various profiles and sizes for gripping mechanism groove 302f can be utilized without changing the scope of the invention.

The disclosed embodiments herein utilize a novel mechanical device that holds a pre-formed knot for easy and quick anastomosis. The embodiments disclosed herein minimize the dependence on a surgeon's dexterity and experience in performing the anastomosis. Thus, novice surgeons will be able learn and produce quality laparoscopic anastomosis in a short time using the disclosed invention.

The system design is simple and therefore it can be mass produced at low cost using existing fabrication techniques.

The design utilizes conventional needles and sutures and therefore does not require custom materials as needed in some prior art.

Manual suturing is still the most widely accepted method of conducting anastomosis, and the similar workflow of the disclosed embodiments will be easily adapted to by surgeons.

Further, the disclosed embodiments have a good market potential that is evident from the fact that even after centuries of technological development, only a handful of automated/assisted anastomosis devices exist in the market. Most of these devices are designed for open surgery and find little or no use in a minimally invasive surgery (MIS) approach. MIS has already become a preferred surgical approach due to its benefits to the patient and it is evident that the number of procedures performed through this approach will increase exponentially in the coming years. At the same time, without any improvement to the laparoscopic anastomosis technique, present anastomosis times of the order of hours will have a huge social and financial burden. Thus an anastomosis device that can potentially reduce anastomosis time from hours to minutes has high market value.

The disclosed embodiments have advantages over existing devices and technologies in terms of their simplicity, close resemblance to manual suturing and low cost. Most of the existing technologies utilize custom designed needles/sutures whereas the disclosed embodiments utilize standardized off-the-shelf needle and sutures. In addition, the mechanical components of the disclosed embodiments can be constructed using materials such as plastics and can be produced at a low cost using standardized manufacturing process (injection molding etc.). The disclosed embodiments are suitable for all types of anastomosis (end-end, end-side and side-side) for vessels with varying diameters.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their to equivalents.

Therefore what is claimed is:

1. An anastomosis device, comprising:
a) an elongate center pin having proximal and distal ends, said proximal end structured to be manipulatable by a manipulation tool, a grip mechanism located at said distal end for gripping a suture;
b) an outer sleeve having a passageway therethrough to receive therein the elongate center pin, said sleeve being mountable by a pre-formed knot formed in a suture; and
c) a suture release mechanism having a passageway extending therethrough to receive the outer sleeve therein, said suture release mechanism being reciprocally translatable on said sleeve between a first position and a second position, and movement of said suture release mechanism from said first position to said second position dislodges said pre-formed knot off said sleeve, and with a free end of said suture being gripped by said grip mechanism, said pre-formed knot is dislodged such that said suture is located within said pre-formed knot and pulling said free end tightens said pre-formed knot on said suture.

2. The anastomosis device according to claim 1 wherein said grip mechanism comprises:
a head section attached to the distal end of said elongate center pin, said head section having a circumferential groove to receive therein a suture,
said elongate center pin including a saw tooth profile spaced from said manipulatable proximal end that includes one or more teeth; and
a locking mechanism located at one end of the outer sleeve, said locking mechanism including a saw-tooth profile that mates with the saw-tooth profile spaced from the proximal end of said elongate center pin, wherein said locking mechanism permits motion of the center pin into the outer sleeve and locks in the opposite direction, and wherein at the end of its travel, the elongate center pin is butted against a distal end of the outer sleeve and is kept in tight contact thereto thereby firmly holding the suture in place.

3. The anastomosis device according to claim 1 wherein said grip mechanism comprises:
a head section attached to the distal end of said elongate center pin; and
a groove disposed on the head section comprising an elongate channel having a width and length and an opening thereto, wherein the opening has a width less than that of the elongate channel, thereby allowing a suture to be inserted into the channel through the opening and to remain firmly held in place in the channel.

4. The anastomosis device according to claim 3 wherein said head section further includes a chamfered opening used to guide a suture towards said groove.

5. The anastomosis device according to claim 2 wherein said suture release mechanism comprises a pair of elongate arms each terminated by a stop attached thereto having a width wider than a width of each of the arms, said locking mechanism including a pair of grooves to receive therein said arms with attached stops, with said grooves having an opening at one end thereof having a width to accept the arms therein but not said stops, such that said suture release mechanism can be translated with respect to the locking mechanism between said first and second positions and is limited in its motion through the use of said grooves in the locking mechanism and said stops on said arms.

6. The anastomosis device according to claim 3 wherein said suture release mechanism comprises a pair of elongate arms each terminated by a stop attached thereto having a width wider than a width of each of the arms, said locking mechanism including a pair of grooves to receive therein said arms with attached stops, with said grooves having an opening at one end thereof having a width to accept the arms therein but not said stops, such that said suture release mechanism can be translated with respect to the locking mechanism between said first and second positions and is limited in its motion through the use of said grooves in the locking mechanism and said stops on said arms.

7. An anastomosis device, comprising:
a) an elongate center pin having proximal and distal ends, said proximal end structured to be manipulatable by a manipulation tool;
b) an outer sleeve having a passageway therethrough to receive therein the elongate center pin, said sleeve being mountable by a pre-formed knot formed in a suture;
c) a suture release mechanism having a passageway extending therethrough to receive the outer sleeve therein, said suture release mechanism being reciprocally translatable on said sleeve between a first position and a second position, and movement of said suture release mechanism from said first position to said second position dislodges said pre-formed knot off said sleeve, and with a free end of said suture being gripped by said grip mechanism, said pre-formed knot is dislodged such that said suture is located within said pre-formed knot and pulling said free end tightens said pre-formed knot on said suture; and
d) a grip mechanism located at said distal end for gripping a suture, the grip mechanism comprising a head section attached to the distal end of said elongate center pin, and a groove disposed on the head section comprising an elongate channel having a width and length and an opening thereto, wherein the opening has a width less than that of the elongate channel, thereby allowing a suture to be inserted into the channel through the opening and to remain firmly held in place in the channel.

8. The anastomosis device according to claim 7 wherein said head section further includes a chamfered opening used to guide a suture towards said groove.

9. The anastomosis device according to claim 7 wherein said suture release mechanism comprises a pair of elongate arms each terminated by a stop attached thereto having a width wider than a width of each of the arms, said locking mechanism including a pair of grooves to receive therein said arms with attached stops, with said grooves having an opening at one end thereof having a width to accept the arms therein but not said stops, such that said suture release mechanism can be translated with respect to the locking mechanism between said first and second positions and is limited in its motion through the use of said grooves in the locking mechanism and said stops on said arms.

10. The anastomosis device according to claim 4 further comprising a suture having a suture needle attached to a first end thereof and a second end of the suture being formed into said pre-formed knot mounted on said outer sleeve.

11. The anastomosis device according to claim 5 further comprising a suture having a suture needle attached to a first end thereof and a second end of the suture being formed into said pre-formed knot mounted on said outer sleeve.

12. The anastomosis device according to claim 6 further comprising a suture having a suture needle attached to a first end thereof and a second end of the suture being formed into said pre-formed knot mounted on said outer sleeve.

13. The anastomosis device according to claim 8 further comprising a suture having a suture needle attached to a first end thereof and a second end of the suture being formed into said pre-formed knot mounted on said outer sleeve.

14. The anastomosis device according to claim 9 further comprising a suture having a suture needle attached to a first end thereof and a second end of the suture being formed into said pre-formed knot mounted on said outer sleeve.
